(19) United States
(12) Patent Application Publication
(10) Pub. No.: US 2004/0062727 A1
(43) Pub. Date: Apr. 1, 2004

(54) Photostable photoprotective UV-screening compositions comprising dibenzoylmethane/methyl-benzylidenecamphor/diarylbutadiene compounds

(75) Inventor: Didier Candau, Bievres (FR)

Correspondence Address: BURNS, DOANE, SWECKER & MATHIS, L.L.P., P.O. Box 1404, Alexandria, VA, 22313-1404, US

(73) Assignee: SOCIETE L'OREAL S.A., Paris (FR)

(21) Appl. No.: 10/463,433

(22) Filed: Jun. 18, 2003

Related U.S. Application Data
(63) Continuation of application No. PCT/FR01/03636, filed on Nov. 20, 2001.

(30) Foreign Application Priority Data
Dec. 18, 2000 (FR) 00/16521

(51) Int. Cl. A61K 7/42

(52) U.S. Cl. 424/59

(57) ABSTRACT

Topically applicable, photostable cosmetic/dermatological UV-screening compositions comprise (a) at least one UV-screening dibenzoylmethane compound, (b) p-methylbenzylidenecamphor, and (c) a methylbenzylidenecamphor photostabilizing amount of at least one 4,4-diarylbutadiene compound, formulated into a topically applicable, cosmetically/dermatologically acceptable support therefor; the weight ratio of the 4,4-diarylbutadiene compound(s) to the dibenzoylmethane compound(s) is characteristically greater than 2.5 and the subject compositions are advantageously devoid of any cinnamate sunscreen.

### CROSS-REFERENCE TO PRIORITY/PCT APPLICATIONS

This application claims priority under 35 U.S.C. § 119 of FR-00/16521, filed Dec. 18, 2000, and is a continuation of PCT/FR01/03636, filed Nov. 20, 2001 and designating the United States (published in the French language on Jun. 27, 2002 as WO 02/49596 A2; the title and abstract were also published in English), both hereby expressly incorporated by reference.

### BACKGROUND OF THE INVENTION

1. Technical Field of the Invention

The invention relates to a cosmetic or dermatological composition for topical application, in particular in a regime or regimen for photoprotecting the skin and the hair, comprising, formulated into a cosmetically acceptable support:

(a) at least one UV-screening agent of the dibenzoylmethane derivative type,

(b) p-methylbenzylidenecamphor, and

(c) at least one 4,4-diarylbutadiene compound; the weight ratio of the 4,4-diarylbutadiene compound to the dibenzoylmethane derivative being greater than 2.5 and said composition characteristically containing no cinnamate sunscreen.

This invention also relates to a process for improving the photostability of p-methylbenzylidene-camphor in the presence of a UV-screening agent of the dibenzoylmethane derivative type, which entails adding thereto an effective amount of at least one 4,4-diaryl-butadiene compound.

2. Description of Background/Related/Prior Art

It is known that light radiation with wavelengths of between 280 nm and 400 nm permit tanning of the human epidermis and that light rays with wavelengths more particularly between 280 nm and 320 nm, known as UV-B rays, cause skin burns and erythema which can harm the development of a natural tan. For these reasons, as well as for aesthetic reasons, there is a constant demand for means of controlling this natural tanning in order thus to control the color of the skin; this UV-B radiation should thus be screened out.

It is also known that UV-A rays, with wavelengths between 320 nm and 400 nm, which cause browning of the skin, are liable to induce adverse changes therein, in particular in the case of sensitive skin or skin which is continually exposed to solar radiation. UV-A rays cause in particular a loss of elasticity of the skin and the appearance of wrinkles leading to premature aging of the skin. They promote triggering of the erythemal reaction or amplify this reaction in certain individuals and may even be the cause of phototoxic or photoallergic reactions. Thus, for aesthetic and cosmetic reasons such as the conservation of the ski's natural elasticity, for example, an increasingly large number of people wish to control the effect of UV-A rays on their skin. It is thus desirable also to screen out UV-A radiation.

In this respect, one particularly advantageous family of UV-A screening agents currently consists of dibenzoylmethane derivatives, and in particular 4-tert-butyl-4′-methoxydibenzoylmethane, which have high intrinsic absorbing power. These dibenzoylmethane derivatives, which are products which are now well known per se as screening agents that are active in the UV-A range, are described in particular in FR-A-2,326,405 and FR-A-2,440,933, as well as in EP-A-0,114,607; 4-tert-butyl-4′-methoxydibenzoylmethane is moreover currently sold under the trademark “Parsol 1789” by Hoffmann LaRoche.

It has already been proposed in patent application FR-2-607,700 to combine p-methylbenzylidenecamphor with 4-tert-butyl-4′-methoxydibenzoylmethane so as to improve the light stability of cosmetic compositions containing 4-tert-butyl-4′-methoxydibenzoylmethane.

However, this solution is not entirely satisfactory, given the fact that p-methylbenzylidene-camphor is itself subject to gradual disappearance under the action of light. Cosmetic compositions containing it undergo a reduction in the concentration of p-methylbenzylidenecamphor under the action of light and thus lose their photoprotective efficacy.

EP-0-967,200, DE-197,46,654, DE-197,55,649, EP-1-008,586, DE-100,07,017, EP-1-133,980 and EP-1-133,981 disclose antisun compositions based on 4,4-diarylbutadienes, which may contain other additional screening agents, for instance 4-tert-butyl-4′-methoxydibenzoylmethane and p-methylbenzylidene.

### SUMMARY OF THE INVENTION

After considerable research conducted in the field of photoprotection mentioned above, it has now unexpectedly and surprisingly been determined that by introducing a 4,4-diarylbutadiene compound into a composition containing p-methylbenzylidenecamphor in combination with at least one dibenzoylmethane derivative, and in particular with 4-tert-butyl-4′-methoxydibenzoylmethane, the photostability of p-methylbenzylidenecamphor in such compositions, and thus the overall efficacy of these compositions, may be considerably improved.

This discovery is the basis of the present invention.

Thus, the present invention features cosmetic or dermatological compositions for topical application, in particular in a regime or regimen for photoprotecting the skin and the hair, comprising, formulated into a cosmetically acceptable support:

(a) at least one UV-screening agent of the dibenzoylmethane derivative type,

(b) p-methylbenzylidenecamphor, and

(c) at least one 4,4-diarylbutadiene compound; the weight ratio of the 4,4-diarylbutadiene compound to the dibenzoylmethane derivative being greater than 2.5 and said composition advantageously being devoid of any cinnamate sunscreen.

The present invention also features a process for improving the photostability of p-methylbenzylidene-camphor in the presence of a UV-screening agent of the dibenzoylmethane derivative type, which entails adding thereto at least one 4,4-diarylbutadiene compound in an effective amount which makes it possible to improve the photostability of p-methylbenzylidenecamphor.

### DETAILED DESCRIPTION OF BEST MODE AND SPECIFIC/PREFERRED EMBODIMENTS OF THE INVENTION

In accordance with the invention, the expression “effective amount of 4,4-diarylbutadiene” means an amount that is sufficient to obtain an appreciable and significant improvement in the photostability of p-methylbenzylidenecamphor of the photoprotective cosmetic composition. This minimum amount of photostabilizing agent to be used, which may vary depending on the nature of the cosmetically acceptable support selected for the composition, may be determined without difficulty by means of a standard test for measuring photostability.

In accordance with the invention, the term “4,4-diarylbutadiene compound” means any molecule comprising at least one 4,4-diarylbutadiene chromophoric group. This molecule may be in the form of a simple compound, an oligomer or a polymer having, on the chain, grafts comprising the chromophoric group.

Thus, according to the present invention, cosmetic compositions may be formulated containing at least one dibenzoylmethane derivative in combination with p-methylbenzylidenecamphor, in which compositions the concentration of p-methylbenzylidenecamphor remains relatively constant even if these compositions are subjected to the action of light.

Other characteristics, aspects, embodiments and advantages of the present invention will become apparent on reading the detailed description that follows.

The compositions of the present invention thus contain p-methylbenzylidenecamphor in combination with at least one dibenzoylmethane derivative.

As indicated above, the dibenzoylmethane derivatives of the present invention are products that are already well known per se and described in particular in FR-2-326,405, FR-2-440,933 and EP-0-114,607, the teachings of which documents are, as regards the actual definition of these products, entirely included as references in the present description.

Among the dibenzoylmethane derivatives which are more particularly targeted by the present invention, mention may be made in particular, in a non-limiting manner, of:

2-methyldibenzoylmethane

4-methyldibenzoylmethane

4-isopropyldibenzoylmethane

4-tert-butyldibenzoylmethane

2,4-dimethyldibenzoylmethane

2,5-dimethyldibenzoylmethane

4,4′-diisopropyldibenzoylmethane

4-tert-butyl-4′-methoxydibenzoylmethane

2-methyl-5-isopropyl-4′-methoxydibenzoylmethane

2-methyl-5-tert-butyl-4′-methoxydibenzoylmethane

2,4-dimethyl-4′-methoxydibenzoylmethane

2,6-dimethyl-4-tert-butyl-4′-methoxydibenzoylmethane.

Among the dibenzoylmethane derivatives mentioned above, it is most particularly preferred to use, according to the present invention, 4-tert-butyl-4′-methoxydibenzoylmethane, in particular the product sold under the trademark “Parsol 1789” by Hoffmann LaRoche, this screening agent corresponding to the structural formula (I) below:

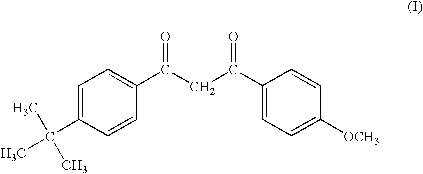

Another dibenzoylmethane derivative which is preferred according to the present invention is 4-isopropyldibenzoylmethane, this screening agent being sold under the name “Eusolex 8020” by Merck and corresponding to the structural formula (II) below:

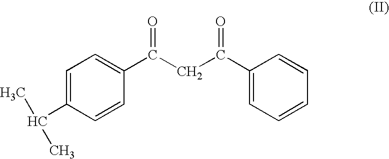

The dibenzoylmethane derivative(s) may be present in the compositions in accordance with the invention in contents preferably ranging from 0.5% to 15% by weight and more preferably from 1% to 10% by weight, relative to the total weight of the composition.

p-Methylbenzylidenecamphor is a UV-B-absorbing liposoluble screening agent that is known per se and is sold especially under the trademark “Eusolex 6300” by Merck. It is present in the compositions in accordance with the invention in contents preferably ranging from 0.5% to 15% by weight and more preferably from 1% to 10% by weight, relative to the total weight of the composition.

Among the preferred 4,4-diarylbutadiene compounds in accordance with the invention that may be selected are the compounds corresponding to formula (III) below:

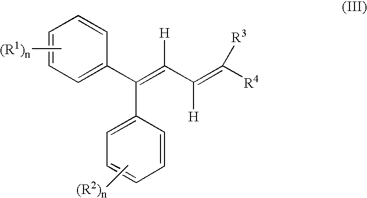

in which the diene system is of Z,Z; Z,E; E,Z or E,E configuration or mixtures of the said configurations, and in which:

R¹ and R², which may be identical or different, denote hydrogen, a linear or branched C₁-C₂₀ alkyl radical; a C₂-C₁₀ alkenyl radical; a C₁-C₁₂ alkoxy radical; a C₃-C₁₀ cycloalkyl radical; a C₃-C₁₀ cycloalkenyl radical; a linear or branched C₁-C₂₀ alkoxycarbonyl radical; a linear or branched C₁-C₁₂ monoalkylamino radical; a linear or branched C₁-C₁₂ dialkylamino radical; an aryl radical; a heteroaryl radical or a hydro-solubilizing substituent chosen from a carboxylate group, a sulfonate group and an ammonium residue;

R³ denotes a group COOR⁵; COR⁵; CONR⁵R⁶; CN; a linear or branched C₁-C₂₀ alkyl radical; a C₂-C₁₀ alkenyl radical; a C₃-C₁₀ cycloalkyl radical; a C₇-C₁₀ bicycloalkyl radical; a C₃-C₁₀ cycloalkenyl radical; a C₇-C₁₀ bicycloalkenyl radical; a C₆-C₁₈ aryl radical; a C₃-C₇ heteroaryl radical;

R⁴ denotes a group COOR⁶; COR⁶; CONR⁵R⁶; CN; a linear or branched C₁-C₂₀ alkyl radical; a C₂-C₁₀ alkenyl radical; a C₃-C₁₀ cycloalkyl radical; a C₇-C₁₀ bicycloalkyl radical; a C₃-C₁₀ cycloalkenyl radical; a C₇-C₁₀ bicycloalkenyl radical; an aryl radical; a heteroaryl radical;

R⁵ and R⁶, which may be identical or different, denote hydrogen; [X]ₚ—R⁷; C₁-C₆-alkylene-SO₃Y; C₁-C₆-alkylene-PO₃Y; C₁-C₆-alkylene-N(R⁸)₃⁺A⁻; a linear or branched C₁-C₂₀ alkyl radical; a C₂-C₁₀ alkenyl radical; a C₃-C₁₀ cycloalkyl radical; a C₇-C₁₀ bicycloalkyl radical; a C₃-C₁₀ cycloalkenyl radical; a C₇-C₁₀ bicycloalkenyl radical; an aryl radical; a heteroaryl radical;

X denotes a group —CH₂—CH₂—Z—, —CH₂CH₂CH₂Z—, —CH(CH₃)—CH₂—Z—, —CH₂—CH₂—CH₂—CH₂—Z— or —CH₂—CH(CH₂CH₃)—Z—;

A denotes Cl, Br, I or SO₄R⁹;

Y denotes hydrogen, Na⁺, K⁺, Mg²⁺, Ca²⁺, Li⁺, Al³⁺ or —N(R⁸)₄⁺

Z denotes O or NH;

R⁷ and R⁸, which may be identical or different, denote hydrogen, a linear or branched C₁-C₆ alkyl radical; a linear or branched C₂-C₆ alkenyl radical; a linear or branched C₁-C₆ acyl radical;

R⁹ denotes hydrogen, a linear or branched C₁-C₆ alkyl radical; a C₂-C₆ alkenyl radical;

n ranges from 1 to 3;

p ranges from 0 to 150.

Examples of C₁-C₂₀ alkyl radicals that may be mentioned are: methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

Examples of C₂-C₁₀alkenyl groups that may be mentioned are: ethenyl, n-propenyl, 1-methylethenyl, n-butenyl, 1-methylpropenyl, 2-methylpropenyl, 1,1-dimethylethenyl, n-pentenyl, 1-methylbutenyl, 2-methylbutenyl, 3-methylbutenyl, 2,2-dimethylpropenyl, 1-ethylpropenyl, n-hexenyl, 1,1-dimethylpropenyl, 1,2-dimethylpropenyl, 1-methylpentenyl, 2-methylpentenyl, 3-methylpentenyl, 4-methylpentenyl, 1,1-dimethylbutenyl, 1,2-dimethylbutenyl, 1,3-dimethylbutenyl, 2,2-dimethylbutenyl, 2,3-dimethylbutenyl, 3,3-dimethylbutenyl, 1-ethylbutenyl, 2-ethylbutenyl, 1,1,2-trimethyl-propenyl, 1,2,2-trimethylpropenyl, propenyl, 1-ethyl-2-methylpropenyl, n-heptenyl, n-octenyl, n-nonenyl, n-decenyl.

As C₁-C₁₂ alkoxy radicals for the radicals R¹ and R², mention may be made of: methoxy, n-propoxy, 1-methylpropoxy, n-pentoxy, 3-methylbutoxy, 2,2-dimethylpropoxy, 1-methyl-1-ethylpropoxy, octoxy, ethoxy, n-propoxy, n-butoxy, 2-methylpropoxy, 1-methylethoxy, 1,1-dimethylpropoxy, hexoxy, heptoxy, 2-ethylhexoxy.

Examples of C₃-C₁₀ cycloalkyl radicals which may be mentioned for the radicals R⁶ and R⁷ are: cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentylcyclopropyl, 1-methyl-1-butylcyclopropyl, 1,2-dimethylcyclopropyl, 1-methyl-2-ethylcyclopropyl, cyclooctyl, cyclononyl or cyclodecyl.

As C₃-C₁₀ cycloalkenyl radicals containing one or more double bonds, for the radicals R⁶ and R⁷, mention may be made of: cyclobutenyl, cyclopentenyl, cyclopentadienyl, cyclohexenyl, 1,3-cyclohexadienyl, 1,4-cyclohexadienyl, cycloheptenyl, cycloheptatrienyl, cyclooctenyl, 1,5-cyclooctadienyl, cyclooctatetraenyl, cyclononenyl or cyclodecenyl.

The cycloalkyl or cycloalkenyl radicals may comprise one or more (preferably from 1 to 3) substituents chosen, for example, from halogen, for instance chlorine, fluorine or bromine; cyano; nitro; amino; C₁-C₄ alkylamino; C₁-C₄ dialkylamino; C₁-C₄ alkyl; C₁-C₄ alkoxy; hydroxyl; they may also comprise from 1 to 3 hetero atoms, for instance sulfur, oxygen or nitrogen, the free valencies of which may be saturated with a hydrogen or a C₁-C₄ alkyl radical.

Examples of acyl radicals that may be mentioned include formyl, acetyl, propionyl and n-butyryl.

The bicycloalkyl or bicycloalkenyl groups are chosen, for example, from bicyclic terpenes, for instance pinane, bornane, pinene, camphor or adamantane derivatives.

The aryl groups are preferably chosen from phenyl and naphthyl rings, which may contain one or more (preferably from 1 to 3) substituents chosen, for example, from halogen, for instance chlorine, fluorine or bromine; cyano; nitro; amino; C₁-C₄ alkylamino; C₁-C₄ dialkylamino; C₁-C₄ alkyl; C₁-C₄ alkoxy; hydroxyl. Phenyl, methoxyphenyl and naphthyl are more particularly preferred.

The heteroaryl groups generally comprise one or more hetero atoms chosen from sulfur, oxygen and nitrogen.

The hydro-solubilizing groups are, for example, carboxylate or sulfonate groups and more particularly salts thereof with physiologically acceptable cations, for instance the alkali metal salts or trialkylammonium salts such as the tris(hydroxyalkyl)ammonium or 2-methyl-1-propanol-2-ammonium salts. Mention may also be made of ammonium groups, for instance alkylammoniums and forms thereof salified with physiologically acceptable anions.

The preferred compounds of formula (III) are chosen from those of formula (IIIa) below:

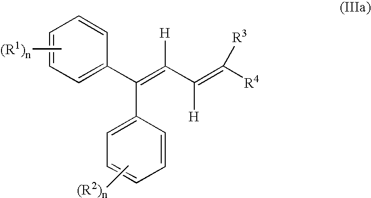

in which the diene system is of Z,Z; Z,E; E,Z or E,E configuration or mixtures of the said configurations, and in which:

R¹ and R², which may be identical or different, denote hydrogen, a C₁-C₈ alkyl radical; a C₁-C₈ alkoxy radical; a hydro-solubilizing substituent chosen from a carboxylate group, a sulfonate group and an ammonium residue;

R³ denotes a group COOR⁵; CONR⁵R⁶; CN;

R⁴ denotes a group COOR⁶; CONR⁵R⁶;

R⁵ denotes hydrogen; [X]ₚ—R⁷; C₁-C₆ alkylene-SO₃Y; C₁-C₆ alkylene-N(R⁸)₃⁺A⁻;

R⁶ denotes [X]ₚ—R⁷; C₁-C₆-alkylene-SO₃Y; C₁-C₆ alkylene-N(R⁸)₃⁺A⁻;

X denotes a —CH₂—CH₂—O—, —CH₂CH₂CH₂O—, —CH(CH₃)—CH₂—O— group;

A denotes Cl, Br, I or SO₄R⁹;

Y denotes hydrogen, Na⁺, K⁺, Mg²⁺, Ca²⁺, Li⁺, Al³⁺ or —N(R⁸)₄⁺

R⁷, R⁸ and R⁹, which may be identical or different, denote hydrogen or a linear or branched C₁-C₃ alkyl radical;

n ranges from 1 to 3;

p ranges from 0 to 50.

The compounds of formula (III) that are even more preferred are chosen from those corresponding to formula (IIIb) below:

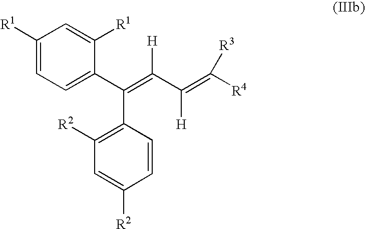

in which the diene system is of Z,Z; Z,E; E,Z or E,E configuration or mixtures of the said configurations, and in which:

R¹ and R², which may be identical or different, denote hydrogen, a C₁-C₈ alkyl radical; a C₁-C₈ alkoxy radical;

R³ denotes a group COOR⁵; CONR⁵R⁶; CN;

R⁴ denotes a group COOR⁶; CONR⁵R⁶;

R⁵ denotes hydrogen; [X]ₚ—R⁷; C₁-C₆-alkylene-SO₃Y; C₁-C₆-alkylene-N(R⁸)₃⁺A⁻;

R⁶ denotes [X]ₚ—R⁷; C₁-C₆-alkylene-SO₃Y; C₁-C₆-alkylene-N(R⁸)₃⁺A⁻;

X denotes a —CH₂—CH₂—O—, —CH₂CH₂CH₂O—, —CH(CH₃)—CH₂—O— group;

A denotes Cl, Br, I or SO₄R⁹;

Y denotes hydrogen, Na⁺, K⁺, Mg²⁺, Ca²⁺, Li⁺, Al³⁺ or —N(R⁸)₄^{'0}

R⁷, R⁸ and R⁹, which may be identical or different, denote hydrogen or a linear or branched C₁-C₃ alkyl radical;

p ranges from 0 to 50.

The compounds of formula (III) that are even more preferred are chosen from those corresponding to formula (IIIc) below:

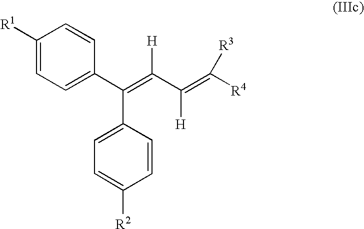

in which the diene system is of Z,Z; Z,E; E,Z or E,E configuration or mixtures of the said configurations, and in which:

R¹ and R², which may be identical or different, denote hydrogen, a C₁-C₈ alkyl radical; a C₁-C₈ alkoxy radical;

R³ denotes a group COOR⁵; CONR⁵R⁶; CN;

R⁴ denotes a group COOR⁶; CONR⁵R⁶;

R⁵ denotes hydrogen; [X]ₚ—R⁷; C₁-C₆-alkylene-SO₃Y; C₁-C₆-alkylene-N(R⁸)₃⁺A⁻;

R⁶ denotes [X]ₚ—R⁷; C₁-C₆-alkylene-SO₃Y; C₁-C₆-alkylene-N(R⁸)₃⁺A⁻;

X denotes a —CH₂—CH₂—O—, —CH₂CH₂CH₂O—, —CH(CH₃)—CH₂—O— group;

A denotes Cl, Br, I or SO₄R⁹;

Y denotes hydrogen, Na⁺, K⁺, Mg²⁺, Ca²⁺, Li⁺, Al³⁺ or —N(R⁸)₄^{'0}

R⁷, R⁸ and R⁹, which may be identical or different, denote hydrogen or a linear or branched C₁-C₃ alkyl radical;

p ranges from 0 to 50.

The compounds of formula (III) that are even more particularly preferred are chosen from the following compounds:

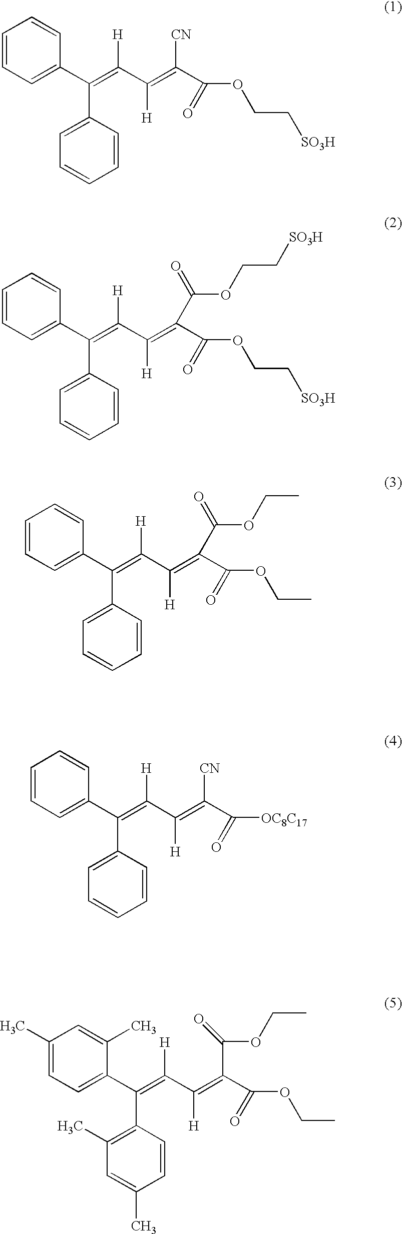

The compounds of formula (III) as defined above are known per se and their structures and syntheses are described in EP-0-967,200, DE-197,46,654 and DE-197,55,649 (which form an integral part of the content of the description).

Among the preferred 4,4-diarylbutadiene compounds in accordance with the invention that may also be mentioned are the oligomers corresponding to formula (IV) below:

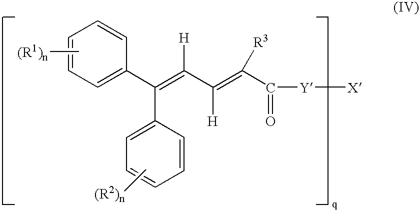

in which the diene system is of Z,Z; Z,E; E,Z or E,E configuration or mixtures of the said configurations, and in which:

R¹, R², R³ and n have the same definitions as in formula (III) above;

Y′ denotes a group —O— or —NR¹⁰—;

R¹⁰ denotes hydrogen; a linear or branched C₁-C₂₀ alkyl radical; a C₂-C₁₀ alkenyl radical; a C₃-C₁₀ cycloalkyl radical; a C₇-C₁₀ bicycloalkyl radical; a C₃-C₁₀ cycloalkenyl radical; a C₇-C₁₀ bicycloalkenyl radical; an aryl radical; a heteroaryl radical;

X′ denotes an aliphatic or cycloaliphatic linear or branched polyol residue comprising from 2 to 10 hydroxyl groups and of valency q; the carbon-based chain of the said residue possibly being interrupted with one or more sulfur or oxygen atoms; one or more imine groups; one or more C₁-C₄ alkylimino groups;

q ranges from 2 to 10.

X′ is a polyol residue containing from 2 to 10 hydroxyl groups and especially:

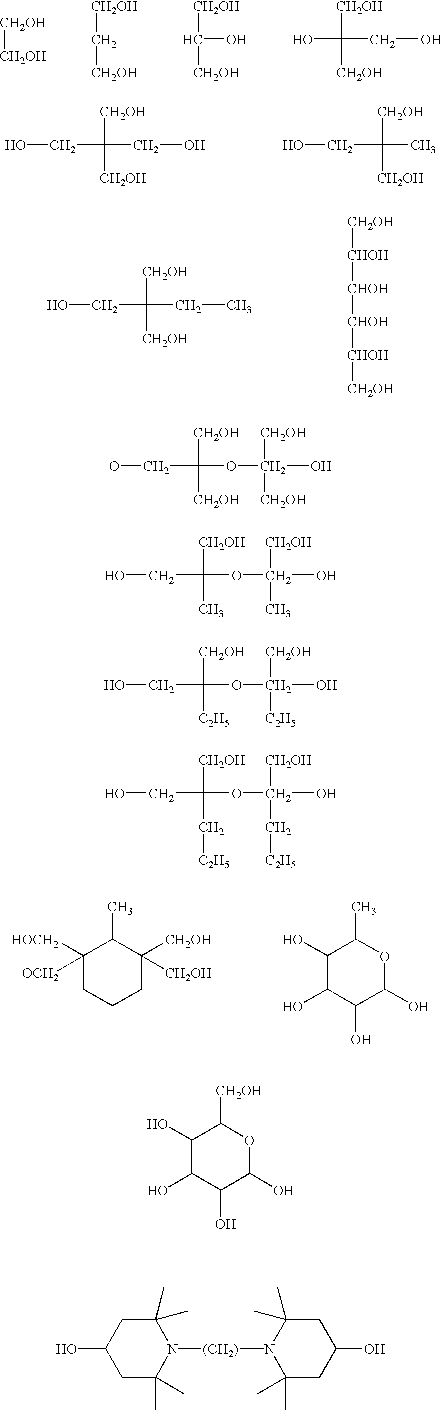

The compounds of formula (IV) that are more preferred are those for which:

R¹ and R², which may be identical or different, denote hydrogen, a C₁-C₁₂ alkyl radical; a C₁-C₈ alkoxy radical; a hydro-solubilizing substituent chosen from a carboxylate group, a sulfonate group and an ammonium residue;

R³ denotes a group COOR⁵; CONR⁵R⁶; CN; a C₃-C₁₀ cycloalkyl radical; a C₇-C₁₀ bicycloalkyl radical;

R⁵ and R⁶, which may be identical or different, denote a linear or branched C₁-C₂₀ alkyl radical; a C₃-C₁₀ cycloalkyl radical; a C₇-C₁₀ bicycloalkyl radical; optionally substituted naphthyl or phenyl;

X′ denotes a polyol residue comprising from 2 to 6 and more particularly from 2 to 4 hydroxyl groups.

The compounds of formula (IV) that are even more preferred are those for which:

X′ denotes an ethanol or pentaerythrol residue.

The compounds of formula (IV) that are even more particularly preferred are chosen from the following compounds:

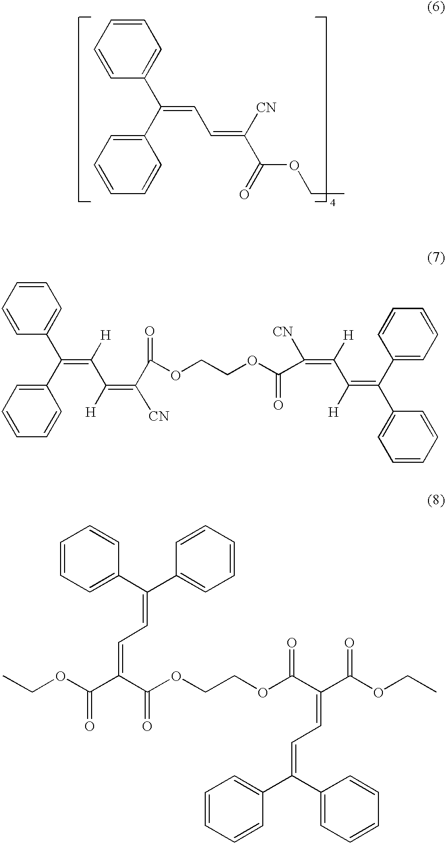

The compounds of formula (IV) as defined above are known per se and their structures and syntheses are described in EP-A-1,008,586 (which forms an integral part of the content of the description).

The 4,4-diarylbutadiene compounds in accordance with the invention are preferably present in the composition of the invention in contents ranging from 0.5% to 15% by weight and more preferably from 1% to 10% by weight, relative to the total weight of the composition.

The compositions in accordance with the invention may also comprise other additional organic UV-screening agents that are active in the UVA and/or UVB range (absorbers), which are water-soluble or liposoluble or even insoluble in the cosmetic solvents commonly used.

The additional organic UV-screening agents are especially chosen from anthranilates; salicylic derivatives; camphor derivatives other than p-methylbenzylidenecamphor; triazine derivatives such as those described in U.S. Pat. No. 4,367,390, EP-863,145, EP-517,104, EP-570,838, EP-796,851, EP-775,698, EP-878,469 and EP-933,376; benzophenone derivatives; β,β-diphenylacrylate derivatives; benzotriazole derivatives; benzimidazole derivatives; imidazolines; bis-benzazolyl derivatives as described in EP-669,323 and U.S. Pat. No. 2,463,264; p-aminobenzoic acid (PABA) derivatives; methylenebis(hydroxyphenyl)-benzotriazole derivatives as described in U.S. Pat. Nos. 5,237,071, 5,166,355, GB-2-303,549, DE-197,26,184 and EP-893,119; screening polymers and screening silicones such as those described especially in WO 93/04665; dimers derived from α-alkylstyrene, such as those described in DE-198,55,649.

As examples of additional UV-A-active and/or UV-B-active organic screening agents, mention may be made of the following, denoted hereinbelow under their INCI name:

Para-Aminobenzoic Acid Derivatives:

PABA,

Ethyl PABA,

Ethyl dihydroxypropyl PABA,

Ethylhexyl dimethyl PABA sold in particular under the name “Escalol 507” by ISP,

Glyceryl PABA,

PEG-25 PABA sold under the name “Uvinul P25” by BASF.

Salicylic derivatives:

Homosalate sold under the name “Eusolex HMS” by Rona/EM Industries,

Ethylhexyl salicylate sold under the name “Neo Heliopan OS” by Haarmann and Reimer,

Dipropylene glycol salicylate sold under the name “Dipsal” by Scher,

TEA salicylate sold under the name “Neo Heliopan TS” by Haarmann and Reimer.

β,β′-Diphenyl Acrylate Derivatives:

Octocrylene sold in particular under the trademark “Uvinul N539” by BASF,

Etocrylene sold in particular under the trademark “Uvinal N35” by BASF,

Benzophenone Derivatives:

Benzophenone-1 sold under the trademark “Uvinul 400” by BASF,

Benzophenone-2 sold under the trademark “Uvinul D50” by BASF,

Benzophenone-3 or Oxybenzone sold under the trademark “Uvinul M40” by BASF,

Benzophenone-4 sold under the trademark “Uvinul MS40” by BASF,

Benzophenone-5,

Benzophenone-6 sold under the trademark “Helisorb 11” by Norquay,

Benzophenone-8 sold under the trademark “Spectra-Sorb UV-24” by American Cyanamid,

Benzophenone-9 sold under the trademark “Uvinul DS-49” by BASF,

Benzophenone-12.

Benzylidenecamphor Derivatives:

b 3-Benzylidenecamphor manufactured under the name “Mexoryl SD” by Chimex,

Benzylidenecamphorsulfonic acid manufactured under the name “Mexoryl SL” by Chimex,

Camphor benzalkonium methosulfate manufactured under the name “Mexoryl SO” by Chimex,

Terephthalylidenedicamphorsulfonic acid manufactured under the name “Mexoryl SX” by Chimex,

Polyacrylamidomethylbenzylidenecamphor manufactured under the name “Mexoryl SW” by Chimex.

Phenyl Benzimidazole Derivatives:

Phenylbenzimidazolesulfonic acid sold in particular under the trademark “Eusolex 232” by Merck,

Benzimidazilate sold under the trademark “Neo Heliopan AP” by Haarmann and Reimer.

Triazine Derivatives:

Anisotriazine sold under the trademark “Tinosorb S” by Ciba Specialty Chemicals,

Ethylhexyl triazone sold in particular under the trademark “Uvinul T150” by BASF,

Diethylhexyl butamido triazole sold under the trademark “Uvasorb HEB”,

2,4,6-tris(Diisobutyl 4′-aminobenzalmalonate)-s-triazine.

Phenyl Benzotriazole Derivatives:

Drometrizole trisiloxane sold under the name “Silatrizole” by Rhodia Chimie,

Methylenebis(benzotriazolyl)tetramethylbutylphenol sold in solid form under the trademark “MIXXIM BB/100” by Fairmount Chemical, or in micronized form as an aqueous dispersion under the trademark “Tinosorb M” by Ciba Specialty Chemicals.

Anthranilic Derivatives:

Menthyl anthranilate sold under the trademark “Neo Heliopan MA” by Haarmann and Reimer.

Inidazoline Derivatives:

Ethylhexyldimethoxybenzylidenedioxoimidazoline propionate.

Benzalmalonate Derivatives:

Polyorganosiloxane containing benzalmalonate functions, sold under the trademark “Parsol SLX” by Hoffmann LaRoche and mixtures thereof.

The organic UV-screening agents that are more particularly preferred are chosen from the following compounds:

Ethylhexyl salicylate,

Octocrylene,

Phenylbenzimidazolesulfonic acid,

Terephthalylidenedicamphorsulfonic acid,

Benzophenone-3,

Benzophenone-4,

Benzophenone-5,

Disodium phenyl dibenzimidazole tetrasulfonate,

Anisotriazine,

Ethylhexyl triazone,

Diethylhexyl butamido triazone,

2,4,6-tris(Diisobutyl 4′-aminobenzalmalonate)-s-triazine,

Methylenebis(benzotriazolyl)tetramethylbutyl-phenol,

Drometrizole trisiloxane, and mixtures thereof.

The cosmetic compositions according to the invention may also comprise pigments or nanopigments (average size of the primary particles: generally between 5 nm and 100 nm and preferably between 10 nm and 50 nm) of coated or uncoated metal oxides, such as, for example, nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are, moreover, alumina and/or aluminum stearate. Such coated or uncoated metal oxide nanopigments are described in particular in EP-A-0-518,772 and EP-A-0-518,773.

The compositions according to the invention may also contain agents for artificially tanning and/or browning the skin (self-tanning agents) such as, for example, dihydroxyacetone (DHA).

The compositions of the invention may also comprise standard cosmetic adjuvants chosen especially from fatty substances, organic solvents, ionic or nonionic thickeners, softeners, antioxidants, free-radical scavengers, opacifiers, stabilizers, emollients, silicones, α-hydroxy acids, antifoams, moisturizers, vitamins, insect repellants, fragrances, preserving agents, surfactants, anti-inflammatories, substance P antagonists, fillers, polymers, propellants, acidifying or basifying agents, colorants or any other ingredient usually used in cosmetics, in particular for the manufacture of antisun compositions in the form of emulsions.

The fatty substances may consist of an oil or a wax or mixtures thereof. The term “oil” means a compound that is liquid at room temperature. The term “wax” means a compound that is solid or substantially solid at room temperature, and whose melting point is generally above 35° C. They also comprise linear or cyclic fatty acids, fatty alcohols and fatty acid esters such as benzoic acid, trimellitic acid and hydroxybenzoic acid derivatives.

Oils that may be mentioned include mineral oils (paraffin); plant oils (sweet almond oil, macadamia oil, blackcurrant pip oil or jojoba oil); synthetic oils, for instance perhydrosqualene, fatty alcohols, fatty acids or fatty esters (for instance the C₁₂-C₁₅ alkyl benzoates sold under the trademark “Finsolv TN” by Finetex, octyl palmitate, isopropyl lanolate, triglycerides including capric/caprylic acid triglycerides), oxyethylenated or oxypropylenated fatty esters and ethers; silicone oils (cyclomethicone, and polydimethylsiloxanes or PDMS) or fluoro oils, and polyalkylenes.

Waxy compounds that may be mentioned include paraffin, carnauba wax, beeswax and hydrogenated castor oil.

Among the organic solvents that may be mentioned are lower alcohols and polyols.

Needless to say, a person skilled in the art will take care to select this or these optional additional compound(s) and/or the amounts thereof such that the advantageous properties, in particular the photostability of the triazine derivative, intrinsically associated with the compositions in accordance with the invention are not, or are not substantially, adversely affected by the envisaged addition(s).

The compositions of the invention may be prepared according to techniques that are well known to those skilled in the art, in particular those intended for preparing emulsions of oil-in-water or water-in-oil type.

These compositions may be in particular in the form of a simple emulsion or a complex emulsion (O/W, W/O, O/W/O or W/O/W), such as a cream, a milk, a gel or a cream-gel; a powder or a solid tube, and may optionally be packaged as an aerosol and may be in the form of a mousse or spray.

When it is an emulsion, the aqueous phase of this emulsion may comprise a nonionic vesicular dispersion prepared according to known processes (Bangham, Standish and Watkins, J. Mol. Biol. 13, 238 (1965), FR-2-315,991 and FR-2-416,008).

The cosmetic composition of the invention may be used as a composition for protecting the human epidermis or the hair against ultraviolet rays, as an antisun composition or as a makeup product.

When the cosmetic composition according to the invention is used for protecting the human epidermis against UV rays, or as an antisun composition, it may be in the form of a suspension or a dispersion in solvents or fatty substances, in the form of a nonionic vesicular dispersion or in the form of an emulsion, preferably of oil-in-water type, such as a milk or a cream, or in the form of an ointment, a gel, a cream-gel, a solid tube, a powder, a stick, an aerosol mousse or a spray.

When the cosmetic composition according to the invention is used in a regime or regimen for protecting the hair against UV rays, it may be in the form of a shampoo, a lotion, a gel, an emulsion or a nonionic vesicular dispersion and may constitute, for example, a rinse-out composition, to be applied before or after shampooing, before or after dyeing or bleaching, or before, during or after permanent-waving or straightening the hair, a styling or treating lotion or gel, a blow-drying or hairsetting lotion or gel, or a permanent-waving, straightening, dyeing or bleaching composition for the hair.

When the composition is used as a makeup product for the eyelashes, the eyebrows or the skin, such as an epidermal treatment cream, a foundation, a tube of lipstick, an eyeshadow, a face powder, mascara or an eyeliner, it may be in solid or pasty, anhydrous or aqueous form, for instance oil-in-water or water-in-oil emulsions, nonionic vesicular dispersions or suspensions.

As a guide, for the antisun formulations in accordance with the invention that contain a support of oil-in-water emulsion type, the aqueous phase (especially comprising the hydrophilic screening agents) generally represents from 50% to 95% by weight and preferably from 70% to 90% by weight, relative to the total weight of the formulation, the oily phase (especially comprising the lipophilic screening agents) from 5% to 50% by weight and preferably from 10% to 30% by weight, relative to the total weight of the formulation, and the (co)emulsifier(s) from 0.5% to 20% by weight and preferably from 2% to 10% by weight, relative to the total weight of the formulation.

As mentioned above, the present invention features the use of a composition as defined above for the manufacture of a cosmetic or dermatological composition intended for protecting the skin and/or the hair against ultraviolet radiation, in particular solar radiation.

The present invention also features a process for improving the stability towards UV radiation of p-methylbenzylidenecamphor in the presence of a UV-screening agent of the dibenzoylmethane derivative type, which entails adding thereto an effective amount of at least one 4,4-diarylbutadiene as defined above.

This present invention also features the use of a UV-screening agent of the 4,4-diarylbutadiene type as defined above in the preparation of a cosmetic or dermatological composition comprising at least one UV-screening agent of the dibenzoylmethane derivative type and at least p-methyl-benzylidenecamphor, with the aim of improving the stability of p-methylbenzylidenecamphor towards UV rays.

In order to further illustrate the present invention and the advantages thereof, the following specific examples are given, it being understood that same are intended only as illustrative and in nowise limitative.

In said examples to follow, all parts and percentages are given by weight, unless otherwise indicated.

The following Tables 1 and 2 report Examples 1 and 2, specific compositions according to the present invention.

TABLE 1

| COMPOSITION | EXAMPLE 1 |
|---|---|
| Glycerol mono/distearate/polyethylene | 2 |
| glycol (100 EO) mixture (Arlacel 165 | |
| FL, ICI) | |
| Stearyl alcohol (Lanette 18, Henkel) | 1 |
| Stearic acid from palm oil (Stéarine | 2.5 |
| TP, Stéarinerie Dubois) | |
| Polydimethylsiloxane (Dow Corning | 0.5 |
| 200 Fluid, Dow Corning) | |
| C₁₂/C₁₅ Alkyl benzoate (Witconol TN, | 20 |
| Witco) | |
| Triethanolamine | 0.5 |
| Octocrylene (Uvinul N539, BASF) | 8 |
| Butylmethoxydibenzoylmethane | 2 |
| (Parsol 1789, Hoffmann-LaRoche) | |
| 4-Methylbenzylidenecampor (Eusolex | 2.5 |
| 6300, Merck) | |
| Compound of formula (1) | 6 |
| Glycerol | 4 |
| Triethanolamine | 0.3 |
| Polyacrylic acid (Synthalen K, 3V) | 0.4 |
| Preservatives | q.s |
| Demineralized water q.s. | 100 g |

TABLE 2

| COMPOSITION | EXAMPLE 2 |
|---|---|
| Glycerol mono/distearate/polyethylene | 2 |
| glycol (100 EO) mixture (Arlacel 165 | |
| FL - ICI) | |
| Stearyl alcohol (Lanette 18, Henkel) | 1 |
| Stearic acid from palm oil (Stéarine | 2.5 |
| TP, Stéarinerie Dubois) | |
| Polydimethylsiloxane (Dow Corning | 0.5 |
| 200 Fluid, Dow Corning) | |
| C₁₂/C₁₅ Alkyl benzoate (Witconol TN, | 20 |
| Witco) | |
| Triethanolamine | 0.5 |
| Butylmethoxydibenzoylmethane | 1 |
| (Parsol 1789, Hoffmann-LaRoche) | |
| Compound of formula (6) | 8 |
| 4-Methylbenzylidenecamphor (Eusolex | 3 |
| 6300, Merck) | |
| Glycerol | 4 |
| Oxybenzone (Uvinul M40, BASF) | 7.5 |
| Triethanolamine | 0.3 |
| Polyacrylic acid (Synthalen K, 3V) | 0.4 |
| Preservatives | q.s |
| Demineralized water q.s. | 100 g |

Each patent, patent application and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable, photostable cosmetic/dermatological UV-screening composition, comprising:
(a) at least one UV-screening dibenzoylmethane compound,
(b) p-methylbenzylidenecamphor, and
(c) a p-methylbenzylidenecamphor photostabilizing amount of at least one 4,4-diarylbutadiene compound, formulated into a topically applicable, cosmetically/dermatologically acceptable support therefor.

2. The photostable cosmetic/dermatological composition as defined by claim 1, the weight ratio of said at least one diarylbutadiene compound to said at least one dibenzoylmethane compound being greater than 2.5 and said composition being devoid of any cinnamate sunscreen.

3. The photostable cosmetic/dermatological composition as defined by claim 1, said at least one dibenzoylmethane compound being selected from the group consisting of:
2-methyldibenzoylmethane,
4-methyldibenzoylmethane,
4-isopropyldibenzoylmethane,
4-tert-butyldibenzoylmethane,
2,4-dimethyldibenzoylmethane,
2,5-dimethyldibenzoylmethane,
4,4′-diisopropyldibenzoylmethane,
4,4′-dimethoxydibenzolymethane,
4-tert-butyl-4′-methoxydibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane,
2,6-dimethyl-4-tert-butyl-4′-methoxydibenzoyl-methane, and mixtures thereof.

4. The photostable cosmetic/dermatological composition as defined by claim 3, said at least one dibenzoylmethane compound comprising 4-(tert-butyl)-4′-methoxydibenzoylmethane.

5. The photostable cosmetic/dermatological composition as defined by Claim 3, said at least one dibenzoylmethane compound comprising 4-isopropyldibenzoylmethane.

6. The photostable cosmetic/dermatological composition as defined by claim 1, said at least one dibenzoylmethane compound comprising from 0.5% to 15% by weight thereof.

7. The photostable cosmetic/dermatological composition as defined by claim 1, said p-methylbenzylidenecamphor comprising from 0.5% to 15% by weight thereof.

8. The photostable cosmetic/dermatological composition as defined by claim 1, said at least one 4,4-diarylbutadiene compound having the formula (III) below: 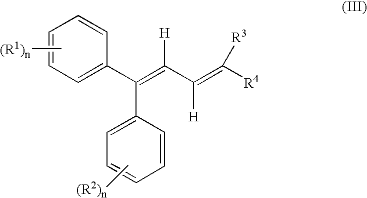
in which the diene system is of Z,Z; Z,E; E,Z or E,E configuration or mixtures of the said configurations, and in which the radicals R¹ and R², which may be identical or different, are each hydrogen, a linear or branched C₁-C₂₀ alkyl radical; a C₂-C₁₀ alkenyl radical; a C₁-C₁₂ alkoxy radical; a C₃-C₁₀ cycloalkyl radical; a C₃-C₁₀ cycloalkenyl radical; a linear or branched C₁-C₂₀ alkoxycarbonyl radical; a linear or branched C₁-C₁₂ monoalkylamino radical; a linear or branched C₁-C₁₂ dialkylamino radical; an aryl radical; a heteroaryl radical or a hydro-solubilizing substituent which comprises a carboxylate group, a sulfonate group or an ammonium residue; R³ is a group COOR⁵; COR⁵; CONR⁵R⁶; CN; a linear or branched C₁-C₂₀ alkyl radical; a C₂-C₁₀ alkenyl radical; a C₃-C₁₀ cycloalkyl radical; a C₇-C₁₀ bicycloalkyl radical; a C₃-C₁₀ cycloalkenyl radical; a C₇-C₁₀ bicycloalkenyl radical; a C₆-C₁₈ aryl radical; a C₃-C₇ heteroaryl radical; R⁴ is a group COOR⁶; COR⁶; CONR⁵R⁶; CN; a linear or branched C₁-C₂₀ alkyl radical; a C₂-C₁₀ alkenyl radical; a C₃-C₁₀ cycloalkyl radical; a C₇-C₁₀ bicycloalkyl radical; a C₃-C₁₀ cycloalkenyl radical; a C₇-C₁₀ bicycloalkenyl radical; an aryl radical; a heteroaryl radical; the radicals R⁵ and R⁶, which may be identical or different, are each hydrogen; [X]ₚ—R⁷; C₁-C₆-alkylene-SO₃Y; C₁-C₆-alkylene-PO₃Y; C₁-C₆-alkylene-N(R⁸)₃⁺A⁻; a linear or branched C₁-C₂₀ alkyl radical; a C₂-C₁₀ alkenyl radical; a C₃-C₁₀ cycloalkyl radical; a C₇-C₁₀ bicycloalkyl radical; a C₃-C₁₀ cycloalkenyl radical; a C₇-C₁₀ bicycloalkenyl radical; an aryl radical; a heteroaryl radical; X is a group —CH₂—CH₂—Z—, —CH₂CH₂CH₂Z—, —CH(CH₃)—CH₂—Z—, —CH₂—CH₂—CH₂—CH₂—Z— or —CH₂—CH(CH₂CH₃)-Z-; A is Cl, Br, I or SO₄R⁹; Y is hydrogen, Na⁺, K⁺, Mg²⁺, Ca²⁺, Li⁺, Al³⁺ or —N(R⁸)₄⁺; Z is O or NH; the radicals R⁷ and R⁸, which may be identical or different, are each hydrogen, a linear or branched C₁-C₆ alkyl radical; a linear or branched C₂-C₆ alkenyl radical; a linear or branched C₁-C₆ acyl radical; R⁹ is hydrogen, a linear or branched C₁-C₆ alkyl radical; a C₂-C₆ alkenyl radical; n ranges from 1 to 3; and p ranges from 1 to 150.

9. The photostable cosmetic/dermatological composition as defined by claim 8, in which the compound of formula (III) has the formula (IIIa) below: 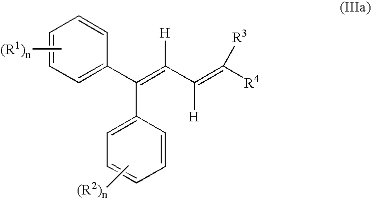
in which the diene system is of Z,Z; Z,E; E,Z or E,E configuration or mixtures of the said configurations, and in which the radicals R¹ and R², which may be identical or different, are each hydrogen, a C₁-C₈, alkyl radical; C₁-C₈ alkoxy radical; a hydro-solubilizing substituent which comprises a carboxylate group, a sulfonate group or an ammonium residue; R³ is a group COOR⁵; CONR⁵R⁶; CN; R⁴ is a group COOR⁶; CONR⁵R⁶; R⁵ is hydrogen; [X]ₚ—R⁷; C₁-C₆-alkylene-SO₃Y; C₁-C₆-alkylene-N(R⁸)₃⁺A⁻; R⁶ is [X]ₚ—R⁷; C₁-C₆-alkylene-SO₃Y; C₁-C₆-alkylene-N(R⁸)₃⁺A⁻; X is a —CH₂—CH₂—O—, —CH₂CH₂CH₂O—, —CH(CH₃)—CH₂—O— group; A is Cl, Br, I or SO₄R⁹; Y is hydrogen, Na⁺, K⁺, Mg²⁺, Ca²⁺, Li⁺, Al³⁺ or —N(R⁸)₄⁺; the radicals R⁷, R⁸ and R⁹, which may be identical or different, are each hydrogen or a linear or branched C₁-C₃ alkyl radical; n ranges from 1 to 3; and p ranges from 1 to 50.

10. The photostable cosmetic/dermatological composition as defined by claim 8, in which the compound of formula (III) has the formula (IIIb) below: 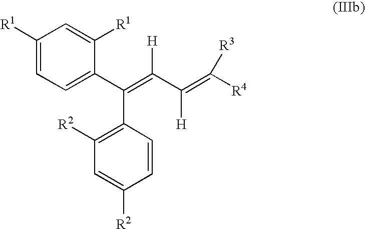
in which the diene system is of Z,Z; Z,E; E,Z or E,E configuration or mixtures of the said configurations, and in which the radicals R¹ and R², which may be identical or different, are each hydrogen, a C₁-C₈ alkyl radical; a C₁-C₈ alkoxy radical; R³ is a group COOR⁵; CONR⁵R⁶; CN; R⁴ is a group COOR⁶; CONR⁵R⁶; R⁵ is hydrogen; [X]ₚ—R⁷; C₁-C₆-alkylene-SO₃Y; C₁-C₆-alkylene-N(R⁸)₃⁺A⁻; R⁶ is [X]ₚ—R⁷; C₁-C₆-alkylene-SO₃Y; C₁-C₆-alkylene-N(R⁸)₃⁺A⁻; X is a —CH₂—CH₂—O—, —CH₂CH₂CH₂O—, —CH(CH₃)—CH₂—O— group; A is Cl, Br, I or SO₄R⁹; Y is hydrogen, Na⁺, K⁺, Mg²⁺, Ca²⁺, Li⁺, Al³⁺ or —N(R⁸)₄⁺; the radicals R⁷, R⁸ and R⁹, which may be identical or different, are each hydrogen or a linear or branched C₁-C₃ alkyl radical; and p ranges from 1 to 50.

11. The photostable cosmetic/dermatological composition as defined by claim 8, in which the compound of formula (III) has the formula (IIIc) below: 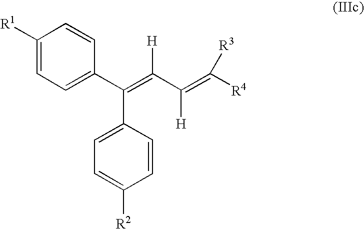
in which the diene system is of Z,Z; Z,E; E,Z or E,E configuration or mixtures of the said configurations, and in which the radicals R¹ and R², which may be identical or different, are each hydrogen, a C₁-C₈ alkyl radical; a C₁-C₈ alkoxy radical; R³ is a group COOR⁵; CONR⁵R⁶; CN; R⁴ is a group COOR⁶; CONR⁵R⁶; R⁵ is hydrogen; [X]ₚ—R⁷; C₁-C₆-alkylene-SO₃Y; C₁-C₆-alkylene-N(R⁸)₃⁺A⁻; R⁶ is [X]ₚ—R⁷; C₁-C₆-alkylene-SO₃Y; C₁-C₆-alkylene-N(R⁸)₃⁺A⁻; X is a —CH₂—CH₂—O—, —CH₂CH₂CH₂O—, —CH(CH₃)—CH₂—O— group; A is Cl, Br, I or SO₄R⁹; Y is hydrogen, Na⁺, K⁺, Mg²⁺, Ca²⁺, Li⁺, Al³⁺ or —N(R⁸)₄⁺; the radicals R⁷, R⁸ and R⁹, which may be identical or different, are each hydrogen or a linear or branched C₁-C₃ alkyl radical; and p ranges from 0 to 50.

12. The photostable cosmetic/dermatological composition as defined by claim 8, in which the 4,4-diarylbutadiene compound is selected from among the following compounds:

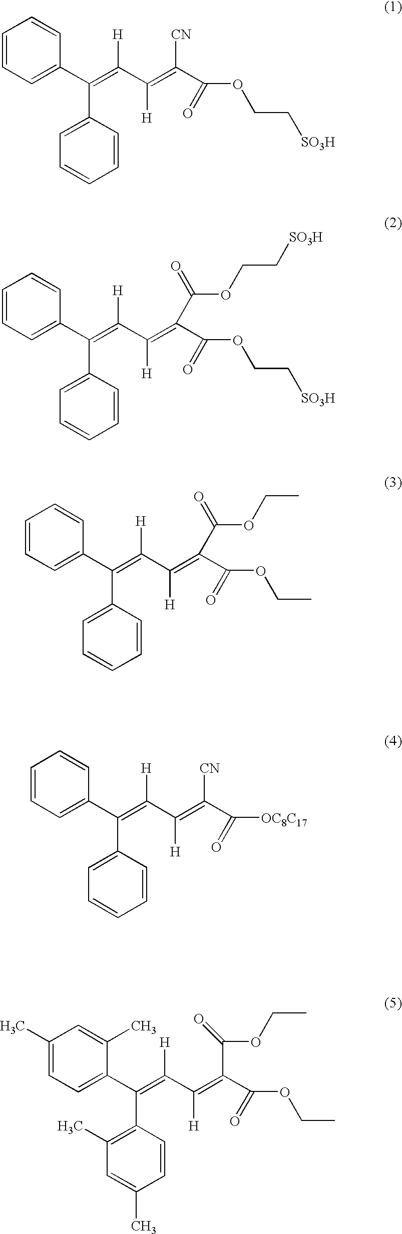

13. The photostable cosmetic/dermatological composition as defined by claim 1, said at least one 4,4-diarylbutadiene compound comprising an oligomer having the formula (IV) below: 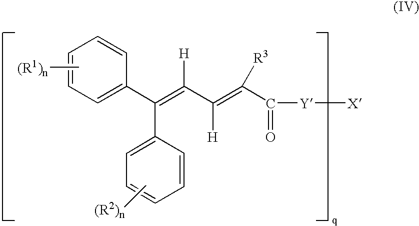
in which the diene system is of Z,Z; Z,E; E,Z or E,E configuration or mixtures of the said configurations, and in which the radicals R¹, R², R³ and n have the same definitions as in formula (III); Y′ is a group —O— or —NR¹⁰—; R¹⁰ is hydrogen; a linear or branched C₁-C₂₀ alkyl radical; a C₂-C₁₀ alkenyl radical; a C₃-C₁₀ cycloalkyl radical; a C₇-C₁₀ bicycloalkyl radical; a C₃-C₁₀ cycloalkenyl radical; a C₇-C₁₀ bicycloalkenyl radical; an aryl radical; a heteroaryl radical; X′ is an aliphatic or cycloaliphatic linear or branched polyol residue comprising from 2 to 10 hydroxyl groups and of valency q; the carbon-based chain of said residue optionally being interrupted with one or more sulfur or oxygen atoms; one or more imine groups; one or more C₁-C₄ alkylimino groups; and q ranges from 2 to 10.

14. The photostable cosmetic/dermatological composition as defined by claim 13, wherein the oligomer of formula (IV), the radicals R¹ and R², which may be identical or different, are each hydrogen, a C₁-C₁₂ alkyl radical; a C₁-C₈ alkoxy radical; a hydro-solubilizing substituent which comprises a carboxylate group, a sulfonate group or an ammonium residue; R³ is a group COOR⁵; CONR⁵R⁶; CN; a C₃-C₁₀ cycloalkyl radical; a C₇-C₁₀ bicycloalkyl radical; the radicals R⁵ and R⁶, which may be identical or different, are each a linear or branched C₁-C₂₀ alkyl radical; a C₃-C₁₀ cycloalkyl radical; a C₇-C₁₀ bicycloalkyl radical; optionally substituted naphthyl or phenyl; X′ is a polyol residue comprising from 2 to 6 hydroxyl groups.

15. The photostable cosmetic/dermatological composition as defined by claim 14, wherein said oligomer of formula (IV), X′ is an ethanol or pentaerythrol residue.

16. The photostable cosmetic/dermatological composition as defined by claim 13, said at least one oligomer of formula (IV) comprising at least one of:

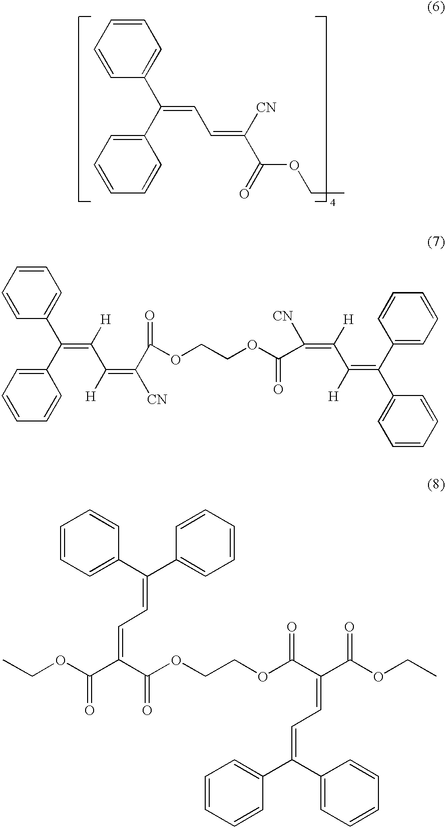

17. The photostable cosmetic/dermatological composition as defined by Claim 1, said at least one 4,4-diarylbutadiene compound comprising from 0.5% to 15% by weight thereof.

18. The photostable cosmetic/dermatological composition as defined by claim 1, further comprising at least one other UV-A-active and/or UV-B-active organic screening agent.

19. The photostable cosmetic/dermatological composition as defined by claim 18, said at least one organic UV-screening agent being selected from the group consisting of anthranilates; salicylic derivatives; camphor derivatives other than p-methylbenzylidenecamphor; triazine derivatives; benzophenone derivatives; β,β′-diphenylacrylate derivatives; benzotriazole derivatives; benzimidazole derivatives; imidazolines; bis-benzazolyl derivatives; p-aminobenzoic acid (PABA) derivatives; methylenebis(hydroxyphenyl)benzotriazole derivatives; screening polymers and screening silicones; dimers derived from α-alkylstyrene, and mixtures thereof.

20. The photostable cosmetic/dermatological composition as defined by claim 19, said at least one other organic UV-screening agent being selected from the group consisting of:
Ethylhexyl salicylate,
Octocrylene,
Phenylbenzimidazolesulfonic acid,
Terephthalylidenedicamphorsulfonic acid,
Benzophenone-3,
Benzophenone-4,
Benzophenone-5,
Disodium phenyl dibenzimidazole tetrasulfonate,
Anisotriazine,
Ethylhexyl triazone,
Diethylhexyl butamido triazone,
2,4,6-tris(Diisobutyl 4′-aminobenzalmalonate)-s-triazine,
Methylenebis(benzotriazolyl)tetramethylbutyl-phenol,
Drometrizole trisiloxane, and mixtures thereof.

21. The photostable cosmetic/dermatological composition as defined by claim 1, further comprising at least one coated or uncoated metal oxide pigment or nanopigment.

22. The photostable cosmetic/dermatological composition as defined by claim 21, said at least one UV-screening pigment or nanopigment comprising titanium oxide, zinc oxide, iron oxide, zirconium oxide, cerium oxide, and mixtures thereof.

23. The photostable cosmetic/dermatological composition as defined by claim 1, further comprising at least one agent for artificially tanning and/or browning the skin.

24. The photostable cosmetic/dermatological composition as defined by claim 1, further comprising at least one adjuvant or additive selected from the group consisting of fatty substances, organic solvents, ionic or nonionic thickeners, softeners, antioxidants, free-radical scavengers, opacifiers, stabilizers, emollients, silicones, α-hydroxy acids, antifoams, moisturizers, vitamins, insect repellants, fragrances, preservatives, agents, surfactants, anti-inflammatories, substance P antagonists, fillers, polymers, propellants, acidifying or basifying agents, colorants, and mixtures thereof.

25. The photostable cosmetic/dermatological composition as defined by Claim 1, formulated for photoprotecting the human epidermis and comprising a nonionic vesicular dispersion, an emulsion, a cream, a milk, a gel, a cream-gel, a suspension, a dispersion, a powder, a solid, a mousse or a spray.

26. The photostable cosmetic/dermatological composition as defined by Claim 1, formulated as a makeup for the eyelashes, the eyebrows or the skin and formulated as solid or pasty, anhydrous or aqueous formulation, or an emulsion, a suspension or a dispersion.

27. The photostable cosmetic/dermatological composition as defined by claim 1, formulated for photoprotecting the hair against ultraviolet rays and comprising a shampoo, a lotion, a gel, an emulsion or a nonionic vesicular dispersion.

28. A regime or regimen for photoprotecting the skin and/or hair against the damaging effects of UV radiation, comprising topically applying thereon an effective amount of the photostable cosmetic/dermatological composition as defined by Claim 1.

29. A method for enhancing the UV photostability of p-methylbenzylidene in the presence of a dibenzoylmethane sunscreen, comprising formulating therewith an effective amount of at least one 4,4-diarylbutadiene compound.
